(19) United States
(12) Patent Application Publication
(10) Pub. No.: US 2017/0112937 A1
(43) Pub. Date: Apr. 27, 2017

(54) METHOD FOR PRODUCING AQUEOUS OPHTHALMIC COMPOSITION, AND AQUEOUS OPHTHALMIC COMPOSITION

(71) Applicant: FUJIFILM CORPORATION, Tokyo (JP)

(72) Inventors: Shirou SONOKE, Kanagawa (JP); Yasuyuki IZUMI, Kanagawa (JP); Shigetomo TSUJIHATA, Kanagawa (JP)

(21) Appl. No.: 15/399,756

(22) Filed: Jan. 6, 2017

Related U.S. Application Data
(63) Continuation of application No. PCT/JP2015/069962, filed on Jul. 10, 2015.

(30) Foreign Application Priority Data
Jul. 11, 2014 (JP) 2014-143640
Mar. 11, 2015 (JP) 2015-048743

(51) Int. Cl. A61K 47/38; A61K 9/00; A61K 9/08; A61K 31/542

(52) U.S. Cl. CPC A61K 47/38; A61K 31/542; A61K 9/0048; A61K 9/08

(57) ABSTRACT

A method of producing an aqueous ophthalmic composition includes wet grinding a mixture that includes a carbonic anhydrase inhibitor, a cellulose derivative, and water, in which a 2%-by-mass aqueous solution of the cellulose derivative has a viscosity of 60 mPa·s or less at 20° C. An aqueous ophthalmic composition includes a carbonic anhydrase inhibitor, a cellulose derivative, and water, in which an absorbance of the aqueous ophthalmic composition at a wavelength of 600 nm and an optical path length of 1 mm is 1.1 or less and a 2%-by-mass aqueous solution of the cellulose derivative has a viscosity of 60 mPa·s or less at 20° C.

### CROSS-REFERENCE TO RELATED APPLICATION

This application is a continuation application of International Application No. PCT/JP2015/069962, filed Jul. 10, 2015, the disclosure of which is incorporated herein by reference in its entirety. Further, this application claims priority from Japanese Patent Application No. 2014-143640, filed Jul. 11, 2014, and Japanese Patent Application No. 2015-048743, filed Mar. 11, 2015, the disclosures of which are incorporated herein by reference in their entirety.

### BACKGROUND OF THE INVENTION

Field of the Invention

The present invention relates to a method of producing an aqueous ophthalmic composition, and an aqueous ophthalmic composition.

Description of the Related Art

Carbonic anhydrase inhibitors used in ophthalmic compositions are generally solid and insoluble or hardly soluble in aqueous solvents such as water that are usually used as ophthalmic carriers. Brinzolamide, which is effective as a carbonic anhydrase inhibitor, is also hardly soluble in water. Therefore, at present, carbonic anhydrase inhibitor-containing ophthalmic compositions are used in the form of a suspension. However, in a case in which a carbonic anhydrase inhibitor-containing ophthalmic composition is used in the form of a suspension, it is concerned that, at the time of the administration thereof to the eye, solid particles in the suspension block light and obstruct the field of vision, temporarily causing a phenomenon of blurred vision.

Further, in the use of such an ophthalmic composition in the form of a suspension, it is also concerned that the administration thereof to the eye may cause irritation to the eye if the solid component has a large particle size. Therefore, it is required that solid particles contained in the suspension be fine.

As a method of preparing a suspension including brinzolamide or the like that is effective as a carbonic anhydrase inhibitor, there has been proposed a method of preparing a suspension in which a solution containing brinzolamide and a surfactant is subjected to wet grinding and a thickening agent such as a carboxyvinyl polymer is incorporated into the resulting suspension (see, for example, International Patent Publication No. WO 2012/053011).

Further, Japanese National-Phase Publication (JP-A) No. 2013-512894, U.S. Pat. No. 6,071,904 and WO 2013/139444 disclose methods that include milling or sizing a carbonic anhydrase inhibitor to adjust the particle size thereof and subsequently mixing the thus processed carbonic anhydrase inhibitor with a solution containing water and a polymer such as hydroxyethyl cellulose. For example, JP-A No. 2013-512894 discloses a method which includes autoclaving a uniformly dispersed slurry containing a carbonic anhydrase inhibitor and a surfactant, sizing the carbonic anhydrase inhibitor particles in the slurry using a microfluidizer, and then mixing this slurry with a polymer slurry containing a polymer and water.

### SUMMARY OF THE INVENTION

In these methods of preparing a carbonic anhydrase inhibitor that are described in WO 2012/053011, JP-A No. 2013-512894, U.S. Pat. No. 6,071,904, and WO 2013/139444, a carbonic anhydrase inhibitor and a surfactant are allowed to coexist and the carbonic anhydrase inhibitor is subjected to grinding or sizing after being heat-treated; however, it is difficult to obtain a suspension containing sufficiently fine particles by any of the methods described in these documents.

Moreover, although it is possible to adjust the viscosity of a suspension by adding thereto an aqueous solution containing a carboxyvinyl polymer, a cellulose derivative and the like to the suspension after the preparation of the suspension, this cannot be expected to micronize solid particles of the carbonic anhydrase inhibitor contained in the suspension and, under the present circumstances, the methods described in the above documents do not effectively inhibit blurred vision that occurs when such a suspension is administered to the eye.

An embodiment of the invention relates to a method of producing an aqueous ophthalmic composition which includes micronized particles of a carbonic anhydrase inhibitor insoluble or hardly soluble in water and is capable of inhibiting blurred vision that occurs when the composition is administered to the eye.

Another embodiment of the invention also relates to an aqueous ophthalmic composition which includes micronized particles of a carbonic anhydrase inhibitor insoluble or hardly soluble in water and is capable of inhibiting blurred vision that occurs when the composition is administered to the eye.

The present invention includes the following embodiments.

[1] A method of producing an aqueous ophthalmic composition, the method comprising wet grinding a mixture that includes a carbonic anhydrase inhibitor, a cellulose derivative, and water, in which a 2%-by-mass aqueous solution of the cellulose derivative has a viscosity of 60 mPa·s or less at 20° C.

[2] The method of producing an aqueous ophthalmic composition according to [1], in which the carbonic anhydrase inhibitor is brinzolamide.

[3] The method of producing an aqueous ophthalmic composition according to [1] or [2], in which the cellulose derivative is at least one of hydroxypropylmethyl cellulose or methyl cellulose.

[4] The method of producing an aqueous ophthalmic composition according to any one of [1] to [3], in which the mixture subjected to the wet grinding further includes a carboxyvinyl polymer.

[5] The method of producing an aqueous ophthalmic composition according to any one of [1] to [4], in which the mixture subjected to the wet grinding further includes a polyoxyethylene fatty acid ester.

[6] The method of producing an aqueous ophthalmic composition according to [5], in which a content of the polyoxyethylene fatty acid ester is from 0.001% by mass to 0.1% by mass with respect to a total mass of the aqueous ophthalmic composition.

[7] The method of producing an aqueous ophthalmic composition according to [5] or [6], in which the polyoxyethylene fatty acid ester is polyoxyethylene monostearate.

[8] The method of producing an aqueous ophthalmic composition according to any one of [1] to [7], in which the mixture subjected to the wet grinding further includes at least one compound selected from the group consisting of sorbic acid and salts thereof.

[9] The method of producing an aqueous ophthalmic composition according to any one of [1] to [8], in which the wet grinding is performed using a bead mill.

[10] The method of producing an aqueous ophthalmic composition according to any one of [1] to [9], in which the method includes adding a diluent containing water to the wet-ground mixture.

[11] The method of producing an aqueous ophthalmic composition according to any one of [1] to [10], in which the method includes subjecting at least some components of the mixture subjected to the wet grinding to moist heat sterilization prior to the wet grinding.

[12] The method of producing an aqueous ophthalmic composition according to [11], in which the components subjected to the moist heat sterilization include the carbonic anhydrase inhibitor, the cellulose derivative, the water, and polyethylene glycol.

[13] An aqueous ophthalmic composition including a carbonic anhydrase inhibitor, a cellulose derivative, and water, in which an absorbance of the aqueous ophthalmic composition at a wavelength of 600 nm and an optical path length of 1 mm is 1.1 or less and a 2%-by-mass aqueous solution of the cellulose derivative has a viscosity of 60 mPa·s or less at 20° C.

[14] The aqueous ophthalmic composition according to [13], in which the carbonic anhydrase inhibitor is brinzolamide.

[15] The aqueous ophthalmic composition according to [13] or [14], in which the cellulose derivative is at least one of hydroxypropylmethyl cellulose or methyl cellulose.

[16] The aqueous ophthalmic composition according to any one of [13] to [15], further including a carboxyvinyl polymer.

[17] The aqueous ophthalmic composition according to any one of [13] to [16], further including a polyoxyethylene fatty acid ester.

[18] The aqueous ophthalmic composition according to [17], in which a content of the polyoxyethylene fatty acid ester is from 0.001% by mass to 0.1% by mass with respect to a total mass of the aqueous ophthalmic composition.

[19] The aqueous ophthalmic composition according to [17] or [18], in which the polyoxyethylene fatty acid ester is polyoxyethylene monostearate.

[20] The aqueous ophthalmic composition according to any one of [13] to [19], further including at least one selected from the group consisting of sorbic acid and salts thereof.

[21] The aqueous ophthalmic composition according to any one of [13] to [20], further including polyethylene glycol.

According to an embodiment of the present disclosure, there can be provided a method of producing an aqueous ophthalmic composition which includes micronized particles of a carbonic anhydrase inhibitor insoluble or hardly soluble in water and is capable of inhibiting blurred vision that occurs when the composition is administered to the eye.

Further, according to another embodiment of the present disclosure, there can be provided an aqueous ophthalmic composition, which includes micronized particles of a carbonic anhydrase inhibitor insoluble or hardly soluble in water and which is capable of inhibiting blurred vision that occurs when the composition is administered to the eye.

### DETAILED DESCRIPTION OF THE INVENTION

Hereinbelow, embodiments of the present disclosure are described.

In the present specification, the term “step” encompasses not only a separate step but also a step which cannot be clearly distinguished from other steps as long as the intended purpose of the step is achieved.

In the present specification, those numerical ranges that are expressed with “to” each denote a range that includes the numerical values stated before and after “to” as the minimum value and the maximum value, respectively.

In the present specification, when reference is made to the amount of a component contained in a composition and there are plural substances corresponding to the same component in the composition, the indicated amount means the total amount of the plural substances present in the composition unless otherwise specified.

Method of Producing Aqueous Ophthalmic Composition

The method of producing an aqueous ophthalmic composition according to the present embodiment includes wet grinding a mixture that includes a carbonic anhydrase inhibitor insoluble or hardly soluble in water, a cellulose derivative whose 2%-by-mass aqueous solution has a viscosity of 60 mPa·s or less at 20° C. (hereinafter, may be referred to as “specific cellulose derivative”), and water (this process may be hereinafter referred to as “wet grinding step”).

If necessary, the method may further include another step such as addition of a diluent containing water to the wet-ground mixture (hereinafter, may be referred to as “dilution step”) or sterilization of components such as raw materials (hereinafter referred to as “sterilization step”).

In the method of producing an aqueous ophthalmic composition according to the present embodiment, when preparing a suspension containing a carbonic anhydrase inhibitor insoluble or hardly soluble in water, a mixture that contains the carbonic anhydrase inhibitor, a cellulose derivative, whose 2%-by-mass aqueous solution has a viscosity of 60 mPa·s or less at 20° C., and water is obtained first before wet grinding and the mixture is then subjected to wet grinding.

It is believed that, in a case in which the carbonic anhydrase inhibitor and the specific cellulose derivative coexist at the time of the wet grinding, the specific cellulose derivative adsorbs to the cross-sections of ground solid-state carbonic anhydrase inhibitor and the surfaces of the ground carbonic anhydrase inhibitor particles are thereby efficiently coated with the specific cellulose derivative.

Since a 2%-by-mass aqueous solution of the cellulose derivative used has a viscosity of 60 mPa·s or less at 20° C., the mixture has a low viscosity and, for example, when grinding of the mixture is performed using a grinding medium, the medium operates efficiently and the dispersion efficiency is thereby improved.

Further, it is thought that the molecules of the cellulose derivative whose 2%-by-mass aqueous solution has a viscosity of 60 mPa·s or less at 20° C. are themselves highly mobile and thus capable of efficiently adsorbing to the ground cross-sections. Thus, it is presumed that, even when the ground particles are fine, reaggregation of the particles is inhibited and particles of a very small size are efficiently generated. It is therefore believed that the particles of the carbonic anhydrase inhibitor contained in the mixture obtained via the wet grinding are very fine and the resulting aqueous ophthalmic composition thus has a low absorbance and excellent transparency, so that blurred vision caused by administration of the composition to the eye is inhibited. However, the functions of the cellulose derivative in the method of producing an aqueous ophthalmic composition are not restricted to those described above.

First, the steps in the method of producing an aqueous ophthalmic composition according to the present embodiment are described. The details of the components used in each step are described hereinafter.

Wet Grinding Step

The wet grinding step is the step of wet grinding a mixture that includes a carbonic anhydrase inhibitor insoluble or hardly soluble in water, a specific cellulose derivative, and water.

Since the carbonic anhydrase inhibitor used in the present embodiment is a solid component insoluble or hardly soluble in water, it is required to be micronized for application to an aqueous ophthalmic composition.

In the present specification, the expression “the carbonic anhydrase inhibitor is a solid component insoluble or hardly soluble in water” means that, when the carbonic anhydrase inhibitor in a free form, which is a solid component, is dissolved in 25° C. water within a neutral pH range of from 6.0 to 8.0, the solubility of the carbonic anhydrase inhibitor in 1 g (1 mL) of water is 10 mg or less at any pH.

The mixture subjected to the wet grinding step includes a carbonic anhydrase inhibitor, a specific cellulose derivative, and water.

Examples of a method of preparing the mixture include a method in which the specific cellulose derivative, and water are thoroughly stirred to dissolve the specific cellulose derivative in the water, and the carbonic anhydrase inhibitor is subsequently added to the resulting solution.

When the specific cellulose derivative is dissolved in water, the water may have normal temperature or may be heated as required.

It is preferable that the resulting aqueous ophthalmic composition is sufficiently sterilized, taking into consideration the safety in the administration thereof to the eye. The sterilization is described below in detail.

The content of the specific cellulose derivative in the mixture is preferably from 10 parts by mass to 300 parts by mass, more preferably from 15 parts by mass to 150 parts by mass, still more preferably from 20 parts by mass to 60 parts by mass, with respect to 100 parts by mass of the carbonic anhydrase inhibitor contained in the mixture.

Wet Grinding

In the wet grinding step, the mixture containing at least the carbonic anhydrase inhibitor, the specific cellulose derivative, and the water is subjected to wet grinding.

In the wet grinding, by allowing the carbonic anhydrase inhibitor and the specific cellulose derivative to coexist in the mixture, a suspension containing fine particles of the carbonic anhydrase inhibitor can be prepared. For example, even if the mixture containing the carbonic anhydrase inhibitor and the water is subjected to wet grinding and then the specific cellulose derivative is added to the resulting suspension, the effects of the present embodiment cannot be obtained sufficiently.

The wet grinding can be performed by an ordinary method. The wet grinding can be performed by, for example, using a known wet grinding apparatus such as a ball mill, a bead mill, a roll mill equipped with plural rolls, a colloid mill, or a cone mill. Further, sizing can also be performed using a high-pressure dispersion apparatus or the like, such as the microfluidizer described in JP-A No. 2013-512891.

Particularly, the wet grinding method is preferably a method using a ball mill or a bead mill, more preferably a method using a bead mill, since the carbonic anhydrase inhibitor having very small particle size can be obtained after the wet grinding.

The bead mill may be any of a batch-type apparatus, a circulation-type apparatus, and a continuous-type apparatus, or a combination thereof. The term “batch-type apparatus” refers to an apparatus in which the whole amount of a liquid to be treated is placed and ground in a bead mill container along with a grinding medium. The term “circulation-type apparatus” refers to an apparatus in which a liquid to be treated is allowed to circulate between a tank and a bead mill container. The term “continuous-type apparatus” refers to an apparatus in which a liquid to be treated is allowed to continuously pass through plural bead mill containers.

The diameter of beads used in the bead mill is preferably from 0.03 mm to 5 mm, more preferably from 0.1 mm to 3 mm, still more preferably from 0.3 mm to 1 mm.

In a case in which the beads used in the bead mill is in this range, the resulting dispersion and the beads can be easily separated after the wet grinding, and the carbonic anhydrase inhibitor particles can be efficiently micronized.

Examples of the type of the beads include glass beads, low-alkali glass beads, alkali-free glass beads, zirconia-silica-based ceramic beads, yttria-stabilized zirconia beads, silicon nitride beads, alumina beads, high-purity alumina beads, and titania beads, among which yttria-stabilized zirconia beads are preferable in terms of its use results in the pharmaceutical production.

It is noted here that yttria-stabilized zirconia beads may be simply referred to as “zirconia beads”.

In the mixture subjected to wet grinding, in addition to the carbonic anhydrase inhibitor, the specific cellulose derivative, and the water, a variety of optional components can be incorporated as desired.

Further, prior to the treatment of the mixture using a wet grinding apparatus, in order to convert the mixture into the form of a slurry suitable for the wet grinding step, for example, water can be added to the mixture in an amount of 5 to 100 times, preferably 5 to 50 times, more preferably 5 to about 25 times the amount of the carbonic anhydrase inhibitor in terms of mass ratio. At the time of the wet grinding, the concentration of the carbonic anhydrase inhibitor is preferably from 0.5 parts by mass to 20 parts by mass, more preferably from 0.8 parts by mass to 12 parts by mass, still more preferably from 1 part by mass to 10 parts by mass, with respect to 100 parts by mass of the mixture to be wet ground.

After connecting a stirring disk to a bead mill apparatus and fitting thereto a bead mill container containing the mixture, the carbonic anhydrase inhibitor that is a solid component is ground into fine particles by performing bead mill dispersion at a rotation speed of preferably from 100 rpm to 10,000 rpm, more preferably from 400 rpm to 6,000 rpm, while cooling the container with a refrigerant, preferably cooling water, at a temperature of preferably from −10° C. to 30° C., more preferably from 2° C. to 15° C.

As a result of performing bead mill dispersion under the above-described conditions, a suspension containing fine particles of the carbonic anhydrase inhibitor can be prepared.

As the bead mill apparatus, any known apparatus can be selected and used as appropriate, as long as the diameter of beads used for dispersion and grinding is in the above-described preferable range.

By performing the wet grinding under the above-described conditions, the solid carbonic anhydrase inhibitor is micronized and, by the function of the coexisting specific cellulose derivative, reaggregation of the resulting particle-form carbonic anhydrase inhibitor is inhibited and fine particles having excellent dispersibility can be obtained.

After the wet grinding, the beads are separated by an ordinary method to obtain a mixture containing fine particles of the carbonic anhydrase inhibitor.

The wet-ground mixture is in the state of a suspension, and this suspension is diluted as required, whereby a suspension having physical properties suitable as an ophthalmic formulation can be prepared.

Dilution Step

If necessary, the thus obtained suspension can be diluted with an addition of a diluent containing at least water to produce an ophthalmic formation.

The diluent may be a liquid consisting of only water, or a liquid containing an optional component such as a viscosity-adjusting agent or a pH-adjusting agent. By using a diluent containing a viscosity-adjusting agent or the like in accordance with the intended purpose, the suspension can be adjusted to have physical properties suitable as an ophthalmic formulation.

Dilution can be performed by adding, as the diluent, water and other component(s) to be contained as required to the wet-ground mixture, preferably after being sterilized.

Sterilization Step

It is preferable that the resulting aqueous ophthalmic composition is sterilized, taking into consideration the safety in the administration thereof to the eye.

For example, respective components used in the production of an aqueous ophthalmic composition are sterilized, and an aqueous ophthalmic composition can be prepared while maintaining the sterile condition. Sterilization may be performed at the stage before mixing each component to be contained in the resulting aqueous ophthalmic composition, or it may be performed in any of the production steps or after the preparation of the aqueous ophthalmic composition, and sterilization may be performed plural times in a combination of these timings.

Hereinbelow, the sterilization step in the method of producing an aqueous ophthalmic composition according to the present embodiment is described.

The carbonic anhydrase inhibitor used in the method of producing an aqueous ophthalmic composition according to the present embodiment is a solid component that is a stable compound. The specific cellulose derivative also has excellent thermal stability; therefore, a variety of sterilization processes can be performed on these components.

Examples of the sterilization processes include sterilization by a dry heat treatment; autoclave sterilization by steam, which is one example of moist heat sterilization; filtration sterilization; plasma sterilization; sterilization using a chemical such as a sterilizing agent; sterilization using a sterilizing gas such as ethylene oxide gas; and sterilization by irradiation of radiation such as gamma-ray. In the use of a chemical or a sterilizing gas for sterilization, the effects of residual components and by-products are concerned. Further, in sterilization by irradiation of radiation, generation of an undesirable degradation product is concerned. Therefore, in terms of the reliability, autoclave sterilization, dry heat sterilization, and filtration sterilization are preferable, and autoclave sterilization is more preferable. The mixture subjected to wet grinding is preferably autoclave-sterilized before being wet ground.

The autoclave sterilization can be performed using an autoclave at a temperature of from 110° C. to 130° C. for a period of from 5 minutes to 60 minutes.

Meanwhile, sterilization of a component having low thermal stability that is decomposed or deteriorated by heat is not done by heating but by, for example, preferably filtration sterilization or long-term heat sterilization at a low temperature, more preferably filtration sterilization. The filtration sterilization can also be performed on a component having excellent thermal stability.

When filtration sterilization is performed, it is preferable to use a sterilization filter having a pore size of 0.2 μm or less. As the sterilization filter, a commercially available product can be employed.

In the method of producing the aqueous ophthalmic composition according to the present embodiment, for the preparation of the mixture to be wet ground, it is preferable to prepare a liquid that contains autoclavable components, namely the carbonic anhydrase inhibitor, the specific cellulose derivative, and the water, along with, as desired, an optional component having excellent thermal stability (hereinafter, this liquid may be referred to as “liquid A”) and to subject this liquid to autoclave sterilization.

In a case in which a component having low thermal stability for which autoclave sterilization is not preferable, such as the below-described sorbic acid or the like, is incorporated into the mixture as a component to be wet ground, a liquid that contains the component having low thermal stability and water (hereinafter, this liquid may be referred to as “liquid B”) can be prepared separately from the liquid A and subjected to filtration sterilization or the like. The liquid B may contain a component having excellent thermal stability as well, or may be a liquid that contains only sterilized water.

Thereafter, a mixture containing the thus sterilized liquids A and B is prepared, and this mixture can be subjected to the wet grinding step.

Further, in a case in which the dilution step is optionally performed, it is preferable that a diluent used in the dilution step (hereinafter, this liquid may be referred to as “liquid C”) is prepared and sterilized in advance.

By wet grinding the above-described mixture that contains the liquids A and B, separating the mixture from the grinding medium, and then diluting the mixture with the liquid C, an aqueous ophthalmic composition having desired physical properties, namely an ophthalmic formulation, can be produced.

The liquid C can be sterilized by a heat sterilization process such as autoclave sterilization.

Optional Step

In addition to the wet grinding step described above and the dilution step performed if required, the method of producing an aqueous ophthalmic composition according to the present embodiment may include an optional step.

Examples of the optional step include the above-described sterilization step; a mixing step for preparing the mixture subjected to the wet grinding step; a coarse dispersion step for homogenizing the mixture subjected to the wet grinding step; and steps performed after the wet grinding step and the dilution step, such as a mixing step for further homogenizing the components contained in the resulting suspension or the like, a dispersion step for imparting a shearing force, a pH-adjusting step, a volume-measuring step, and a filling step of filling the resulting aqueous composition into an arbitrary container.

By the method of producing an aqueous ophthalmic composition according to the present embodiment that includes the above-described steps, an aqueous ophthalmic composition which includes micronized particles of a carbonic anhydrase inhibitor and is capable of inhibiting blurred vision can be obtained.

Absorbance of Aqueous Ophthalmic Composition

The aqueous ophthalmic composition obtained by the method of producing an aqueous ophthalmic composition according to the present embodiment is a suspension in which carbonic anhydrase inhibitor particles contained therein are fine and reaggregation of the particles is inhibited.

As a standard for micronization of the carbonic anhydrase inhibitor particles, the absorbance of the aqueous ophthalmic composition at a wavelength of 600 nm and an optical path length of 1 mm is preferably 1.1 or less, more preferably 0.7 or less, still more preferably 0.4 or less.

In the aqueous ophthalmic composition obtained by the method of producing an aqueous ophthalmic composition according to the present embodiment, since the carbonic anhydrase inhibitor particles are sufficiently micronized, the absorbance of the aqueous ophthalmic composition is controlled to be 1.1 or less and blurred vision after the administration of the composition to the eye can be effectively inhibited.

The lower limit value of the absorbance is not particularly restricted, and it may be higher than 0.0001.

As described above, the absorbance in the present specification is one which is measured at a wavelength of 600 nm and corresponds to an optical path length of 1 mm. However, for example, in a case in which it is difficult to measure the absorbance at an optical path length of 1 mm due to the physical properties of the aqueous ophthalmic composition such as viscosity, the absorbance may be measured after diluting the aqueous ophthalmic composition with water. When the absorbance of the aqueous ophthalmic composition is measured after dilution with water, the value measured at an optical path length obtained by multiplying 1 mm by the dilution factor is defined as the absorbance at an optical path length of 1 mm. For instance, when the absorbance is measured after 10-fold dilution of the composition, the value is measured at an optical path length of 10 mm. Here, the dilution of the composition is performed at a factor of 1 to 10 in terms of volume ratio. The optical path length is measured at 25° C.

Viscosity of Aqueous Ophthalmic Composition

The viscosity of the aqueous ophthalmic composition obtained by the method of producing an aqueous ophthalmic composition according to the present embodiment is preferably in a range of from 10 mPa·s to 200 mPa·s, more preferably in a range of from 20 mPa·s to 100 mPa·s, at 25° C.

In a case in which the viscosity of the aqueous ophthalmic composition is in this range, it is preferable since the aqueous ophthalmic composition can be comfortably administered to the eye and favorable retention on the eyeball surface can be provided when the aqueous ophthalmic composition is administered to the eye.

The viscosity of the aqueous ophthalmic composition can be measured by the method described in “The Japanese Pharmacopoeia 16th Edition”.

Aqueous Ophthalmic Composition

The aqueous ophthalmic composition of the present embodiment is the aqueous ophthalmic composition including the carbonic anhydrase inhibitor, the cellulose derivative, and the water, in which the absorbance of the aqueous ophthalmic composition at a wavelength of 600 nm and an optical path length of 1 mm is 1.1 or less and a 2%-by-mass aqueous solution of the cellulose derivative has a viscosity of 60 mPa·s or less at 20° C.

Components Contained in Aqueous Ophthalmic Composition

Hereinbelow, the components used for the production of an aqueous ophthalmic composition in the method of producing an aqueous ophthalmic composition according to the present embodiment and the components contained in the aqueous ophthalmic composition of the present embodiment are each described in detail.

Carbonic Anhydrase Inhibitor

The carbonic anhydrase inhibitor is not particularly restricted as long as it is one which is a solid component insoluble or hardly soluble in water.

As described above, when the carbonic anhydrase inhibitor in a free form, which is insoluble or hardly soluble in water, is dissolved in 25° C. water within a neutral pH range of from 6.0 to 8.0, the solubility in 1 g of water, that is, 1 mL of water, is 10 mg or less at any pH.

Examples of the carbonic anhydrase inhibitor used in the production of an aqueous ophthalmic composition in the present embodiment include brinzolamide, dorzolamide, acetazolamide, and methazolamide, all of which may be in the form of a salt.

Brinzolamide, dorzolamide, acetazolamide, and methazolamide all have a solubility of 10 mg or less in 1 g of 25° C. water at any pH within a neutral range of from 6.0 to 8.0.

In a case in which the carbonic anhydrase inhibitor forms a salt, the salt is not particularly restricted as long as it is a salt that is usually used as a medicine. Examples of the salt formed by the carbonic anhydrase inhibitor include salts with inorganic acids, salts with organic acids, quaternary ammonium salts, salts with halogen ions, salts with alkali metals, salts with alkaline earth metals, metal salts, ammonia salt, and salts with organic amines.

Examples of the salts with inorganic acids include salts with hydrochloric acid, hydrobromic acid, hydroiodic acid, nitric acid, sulfuric acid, or phosphoric acid.

Examples of the salts with organic acids include salts with acetic acid, oxalic acid, fumaric acid, maleic acid, succinic acid, citric acid, tartaric acid, adipic acid, gluconic acid, glucoheptonic acid, glucuronic acid, terephthalic acid, methanesulfonic acid, lactic acid, hippuric acid, 1,2-ethanedisulfonic acid, isethionic acid, lactobionic acid, oleic acid, pamoic acid, polygalacturonic acid, stearic acid, tannic acid, trifluoromethanesulfonic acid, benzenesulfonic acid, p-toluenesulfonic acid, lauryl sulfate, methyl sulfate, naphthalenesulfonic acid, or sulfosalicylic acid.

Examples of the quaternary ammonium salts include salts with methyl bromide or methyl iodide.

Examples of the salts with halogen ions include salts with a bromide ion, a chloride ion, or an iodide ion.

Examples of the salts with alkali metals include salts with lithium, sodium, or potassium. Examples of the salts with alkaline earth metals include salts with calcium or magnesium.

Examples of the metal salts include salts with iron or zinc.

Examples of the salts with organic amines include salts with triethylenediamine, 2-aminoethanol, 2,2-iminobis(ethanol), 1-deoxy-1-(methylamino)-2-D-sorbitol, 2-amino-2-(hydroxymethyl)-1,3-propanediol, procaine, or N,N-bis(phenylmethyl)-1,2-ethanediamine.

The carbonic anhydrase inhibitor contained in the aqueous ophthalmic composition of the present embodiment is preferably at least one of brinzolamide or dorzolamide, more preferably brinzolamide.

Brinzolamide can be used in the form of a salt; however, in terms of its use results and of achieving favorable pharmacological effects, brinzolamide is preferably used in a free form that does not constitute a salt.

The aqueous ophthalmic composition of the present embodiment may contain only one carbonic anhydrase inhibitor, or two or more carbonic anhydrase inhibitors.

Here, unless otherwise specified, the content of each component shown below is based on the amount of the component with respect to the total amount of the aqueous ophthalmic composition administered to the eye.

In terms of achieving sufficient effect, the content of the carbonic anhydrase inhibitor with respect to the total mass of the aqueous ophthalmic composition is, in terms of the total amount, preferably from 0.1% by mass to 10% by mass, more preferably from 0.2% by mass to 5% by mass, still more preferably from 0.5% by mass to 2% by mass. The content of the carbonic anhydrase inhibitor is the amount in terms of free-form carbonic anhydrase inhibitor.

Cellulose Derivative

The aqueous ophthalmic composition of the present embodiment includes a specific cellulose derivative.

As the cellulose derivative, any cellulose derivative can be used with no particular restriction, as long as the viscosity of a 2%-by-mass aqueous solution of the cellulose derivative at 20° C. is 60 mPa·s or less.

The viscosity of the 2%-by-mass aqueous solution of the cellulose derivative at 20° C. is more preferably 30 mPa·s or less, still more preferably 7 mPa·s or less. The lower limit value of the viscosity is not particularly restricted; however, in terms of the effect, it is preferably 1 mPa·s or higher.

The 2%-by-mass aqueous solution of the cellulose derivative can be prepared by an ordinary method. For example, an aqueous solution of the specific cellulose derivative can be prepared by adding weighed cellulose derivative to water such as ion-exchanged water or pure water and sufficiently stirring the resultant.

In the present specification, as the viscosity of the 2%-by-mass aqueous solution of the cellulose derivative at 20° C., a value measured by the method described in “The Japanese Pharmacopoeia 16th Edition” is used.

Examples of a specific cellulose derivative that can be used in the aqueous ophthalmic composition of the present embodiment include hydroxypropylmethyl cellulose, hydroxypropyl cellulose, hydroxyethyl cellulose, hydroxyethylmethyl cellulose, methyl cellulose, ethyl cellulose, carboxymethyl cellulose, and hydroxypropylmethyl cellulose phthalate. Among these, in terms of the effect, hydroxypropylmethyl cellulose, hydroxypropyl cellulose, hydroxyethyl cellulose, hydroxyethylmethyl cellulose, methyl cellulose, and ethyl cellulose are preferable, and hydroxypropylmethyl cellulose and methyl cellulose are more preferable.

In the present embodiment, the aqueous ophthalmic composition may contain only one specific cellulose derivative, or two or more specific cellulose derivatives.

In the method of producing an aqueous ophthalmic composition according to the present embodiment, the specific cellulose derivative is incorporated in an amount of preferably from 10 parts by mass to 300 parts by mass, more preferably from 15 parts by mass to 150 parts by mass, still more preferably from 20 parts by mass to 60 parts by mass, with respect to 100 parts by mass of the carbonic anhydrase inhibitor contained in the mixture.

Further, the content of the specific cellulose derivative with respect to the total mass of the aqueous ophthalmic composition of the present embodiment and an aqueous ophthalmic composition obtained by the method of producing an aqueous ophthalmic composition according to the present embodiment is, in terms of the total amount, preferably from 0.1% by mass to 3% by mass, more preferably from 0.15% by mass to 1.5% by mass.

Optional Components in Aqueous Ophthalmic Composition

Hereinbelow, components other than the carbonic anhydrase inhibitor and specific cellulose derivative, which can be incorporated into the aqueous ophthalmic composition of the present embodiment and an aqueous ophthalmic composition obtained by the method of producing an aqueous ophthalmic composition according to the present embodiment, are described.

Carboxyvinyl Polymer

It is preferable that the aqueous ophthalmic composition of the present embodiment contains a carboxyvinyl polymer. In a case in which the carboxyvinyl polymer is contained, the viscosity of the composition is appropriately adjusted, so that favorable retention on the eyeball surface can be provided when the aqueous ophthalmic composition is administered to the eye. Further, in a case in which the mixture subjected to wet grinding includes the carboxyvinyl polymer, the carboxyvinyl polymer functions as a dispersant and the speed of the wet grinding process can thereby be improved.

The carboxyvinyl polymer is preferably one whose 0.5%-by-mass aqueous solution has a viscosity of from 4,000 mPa·s to 40,000 mPa·s at 25° C. and pH 7.5. Examples of commercially available carboxyvinyl polymers that can be used in the aqueous ophthalmic composition include CARBOPOL (registered trademark) 971PNF, CARBOPOL (registered trademark) 974PNF, and CARBOPOL (registered trademark) 71GNF (all of which are manufactured by The Lubrizol Corporation), among which CARBOPOL (registered trademark) 974PNF and CARBOPOL (registered trademark) 971PNF are preferable in terms of the solubility.

The viscosity of the 0.5%-by-mass aqueous solution of the carboxyvinyl polymer can be measured by the method described in “Japanese Pharmaceutical Excipients 2013”.

In the method of producing an aqueous ophthalmic composition according to the present embodiment, the carboxyvinyl polymer can be added in any step. However, it is preferable that the carboxyvinyl polymer is contained in the mixture subjected to wet grinding or in the diluent used in the dilution step, and it is more preferable that the carboxyvinyl polymer is contained in both the mixture subjected to wet grinding and the diluent used in the dilution step.

The content of the carboxyvinyl polymer is preferably from 0.1% by mass to 10% by mass, more preferably from 0.2% by mass to 5% by mass, still more preferably from 0.3% by mass to 1% by mass, with respect to the total mass of the aqueous ophthalmic composition.

Further, the carboxyvinyl polymer is contained in an amount of preferably from 0.1 parts by mass to 50 parts by mass, more preferably from 1 part by mass to 20 parts by mass, still more preferably from 3 parts by mass to 10 parts by mass, with respect to 100 parts by mass of the carbonic anhydrase inhibitor contained in the mixture subjected to wet grinding.

Polyoxyethylene Fatty Acid Ester

A polyoxyethylene fatty acid ester is useful for further micronizing the particles of the carbonic anhydrase inhibitor contained in the aqueous ophthalmic composition and more effectively inhibiting blurred vision caused by administration of the composition to the eye.

Examples of the polyoxyethylene fatty acid ester include polyoxyethylene monostearates (polyoxyl stearates) such as polyoxyl 40 stearate, polyoxyl 45 stearate, or polyoxyl 55 stearate, among which polyoxyl 40 stearate is preferable in terms of its use results as an eye drop.

The aqueous ophthalmic composition may contain only one polyoxyethylene fatty acid ester, or two or more polyoxyethylene fatty acid esters.

In the method of producing an aqueous ophthalmic composition according to the present embodiment, it is preferable that the polyoxyethylene fatty acid ester is contained in the mixture subjected to wet grinding.

The content of the polyoxyethylene fatty acid ester is preferably from 0.001% by mass to 0.1% by mass, more preferably from 0.01% by mass to 0.05% by mass, with respect to the total mass of the aqueous ophthalmic composition.

Sorbic Acid and Salts Thereof

In a case in which at least one selected from the group consisting of sorbic acid and salts thereof is incorporated, the size of the carbonic anhydrase inhibitor particles can be made even smaller, so that the absorbance can be reduced and blurred vision caused by the administration of the aqueous ophthalmic composition to the eye can be more effectively inhibited.

Examples of the salts of sorbic acid include sodium salt of sorbic acid and potassium salt of sorbic acid and, in terms of its use results as an eye drop, potassium salt of sorbic acid is preferable.

Although sorbates, particularly potassium sorbate, are known as preservatives, it is a novel finding made by the present inventors that the use of a sorbic acid salt in the wet grinding process in combination with materials to be ground is useful for micronization of the carbonic anhydrase inhibitor.

In the method of producing an aqueous ophthalmic composition according to the present embodiment, at least one selected from the group consisting of sorbic acid and salts thereof may be incorporated into the mixture subjected to wet grinding. In the preparation of the mixture, in terms of thermal stability, it is preferable that the at least one compound selected from the group consisting of sorbic acid and salts thereof is contained in the liquid B.

The content of the compound selected from the group consisting of sorbic acid and salts thereof is preferably from 0.01% by mass to 0.1% by mass, more preferably from 0.03% by mass to 0.05% by mass, with respect to the total mass of the aqueous ophthalmic composition.

Surfactant

Any known surfactant may be used without particular limitation as long as it is applicable to aqueous ophthalmic compositions, shows favorable biocompatibility without causing irritation, and can improve the dispersion stability of solid particles.

Examples of such a surfactant include anionic surfactants, cationic surfactants, amphoteric surfactants, and non-ionic surfactants, among which non-ionic surfactants are preferable.

Examples of the non-ionic surfactants include alkylaryl polyether alcohol polymers such as tyloxapol; polyoxyethylene-polyoxypropylene polymers (poloxamers) such as PLURONIC (trade name, manufactured by BASF Corp.) or LUTROL (trade name, manufactured by BASF Corp.); polyoxyethylene alkylphenyl ethers such as TRITON X-100 (trade name, manufactured by The Dow Chemical Company); polyoxyethylene fatty acid esters such as polyoxyethylene monostearate (also referred to as “polyoxyl stearate”); polyoxyethylene sorbitan fatty acid esters such as polyoxyethylene sorbitan monooleate, polyoxyethylene sorbitan monolaurate, polyoxyethylene sorbitan monopalmitate, or polyoxyethylene sorbitan monostearate; polyoxyethylene castor oils such as polyoxyl 35 castor oil; polyoxyethylene hydrogenated castor oils; sorbitan fatty acid esters such as sorbitan monooleate, sorbitan monolaurate, sorbitan monopalmitate, or sorbitan monostearate; polyoxyethylene alkyl ethers such as polyoxyethylene lauryl ether; polyoxyethylene fatty acid esters such as polyoxyethylene monostearate; and mixtures thereof.

The surfactant has favorable thermal stability, and therefore, can be incorporated into a liquid containing the carbonic anhydrase inhibitor, the specific cellulose derivative, and water (liquid A) in the preparation of the mixture.

In a case in which the surfactant is used, the content thereof is preferably from 0.005% by mass to 1.0% by mass, with respect to the total mass of the aqueous ophthalmic composition.

Isotonizing Agent

Any isotonizing agent that is commonly used in eye drops, such as sodium chloride, glycerol, glucose, mannitol, or sorbitol, may be used. Among these, sodium chloride is preferable as an isotonizing agent because it shows excellent dispersion when made into a formulation, inhibits the formation of aggregates, and provides a composition having excellent redispersibility. It is preferable that the isotonizing agent is added in such an amount that allows the resulting aqueous ophthalmic composition to have an osmotic pressure equivalent to that of tears, specifically an osmotic pressure ratio (ratio of osmotic pressure with respect to physiological saline) in a range of from 0.9 to 1.2.

Since the isotonizing agent is used for adjusting the final physical properties of the aqueous ophthalmic composition, it is preferable that the isotonizing agent is contained in the diluent (liquid C).

Buffer

Any buffer may be used without particular limitation as long as it is a compound capable of buffering a composition in a pH range of from 4 to 10.

Examples of such a buffer include acetates such as sodium acetate; phosphates such as sodium dihydrogen phosphate, disodium hydrogen phosphate, potassium dihydrogen phosphate, or dipotassium hydrogen phosphate; ε-aminocaproic acid; amino acid salts such as sodium glutamate; boric acid and salts thereof; and mixtures thereof.

pH-Adjusting Agent

A pH-adjusting agent such as hydrochloric acid, citric acid, phosphoric acid, acetic acid, tartaric acid, sodium hydroxide, potassium hydroxide, sodium carbonate, or sodium bicarbonate may be used.

The pH of the aqueous ophthalmic composition is adjusted preferably in a range of from 4 to 10 in which irritation to the ocular mucous is generally less, more preferably in a range of from 6 to 8.

Chelating Agent

A chelating agent such as disodium edetate, trisodium edetate, tetrasodium edetate, diethyleneamine pentaacetate, or a mixture thereof may be used. Among these, disodium edetate is preferable.

The content of the chelating agent is preferably from 0.001% by mass to 0.1% by mass, with respect to the total mass of the aqueous ophthalmic composition.

The buffer, pH-adjusting agent, and chelating agent are used for adjusting the final physical properties of the aqueous ophthalmic composition; therefore, in the method of producing an aqueous ophthalmic composition according to the present embodiment, it is preferable that the buffer, pH-adjusting agent, and chelating agent are contained in the diluent (liquid C).

Antioxidant

An antioxidant, such as ascorbic acid; an ascorbate such as sodium ascorbate; tocopherol; or a sulfite such as sodium sulfite, potassium sulfite, magnesium sulfite, calcium sulfite, sodium bisulfite, potassium bisulfite, magnesium bisulfite, calcium bisulfite, sodium metabisulfite, potassium metabisulfite, calcium metabisulfite, sodium thiosulfate, or sodium hydrogen sulfite, may be used.

Preservative

In a case in which a preservative is incorporated, contamination with microorganisms such as fungi or bacteria can be prevented.

As the preservative, a compound which has antibacterial and antifungal effects, applicable to the eye, and exhibits favorable biocompatibility and suppressed irritation can be selected and used.

Examples of such a preservative include quaternary ammonium salts such as benzalkonium chloride or benzethonium chloride; cationic compounds such as chlorhexidine gluconate; p-hydroxybenzoates such as methyl p-hydroxybenzoate, ethyl p-hydroxybenzoate, propyl p-hydroxybenzoate, or butyl p-hydroxybenzoate; alcohol compounds such as chlorobutanol or benzyl alcohol; sodium dehydroacetate; thiomersal; and mixtures thereof.

Among these, quaternary ammonium salts are preferable because they prevent the aggregate formation of fine particles of the carbonic anhydrase inhibitor and inhibit a reduction in pH, and provide a composition having excellent redispersibility and stability. As such quaternary ammonium salts, benzalkonium chloride and benzethonium chloride are more preferable.

The content of the preservative is preferably in a range of from 0.001% by mass to 0.05% by mass, more preferably in a range of from 0.002% by mass to 0.01% by mass, with respect to the total mass of the aqueous ophthalmic composition.

Since the preservative is used for adjusting the final physical properties of the aqueous ophthalmic composition, it is preferable that the preservative is contained in the diluent (liquid C).

Other Components

In the aqueous ophthalmic composition, a polyethylene glycol (MACROGOL) and the like may be incorporated for the purposes of adjusting the viscosity, improving the stability of the aqueous ophthalmic composition, and improving the productivity.

The polyethylene glycol is preferably at least one of MACROGOL 4000 or MACROGOL 6000 in terms of its use results as an eye drop.

In the method of producing an aqueous ophthalmic composition according to the present embodiment, as described above, it is preferable that the mixture containing the carbonic anhydrase inhibitor, the cellulose derivative, and the water is subjected to moist heat sterilization before being subjected to wet grinding. It is also preferable to perform moist heat sterilization on the mixture that contains the polyethylene glycol in addition to the carbonic anhydrase inhibitor, the cellulose derivative, and the water, because this further improves the dispersibility of the aggregates of carbonic anhydrase inhibitor particles when coarse dispersion is subsequently performed for homogenizing the mixture subjected to the wet grinding step.

Sterilization of Optional Components

The components used in the aqueous ophthalmic composition are each sterilized in accordance with the properties of the respective components and the timing of their addition in the production process.

In the aqueous ophthalmic composition of the present embodiment and an aqueous ophthalmic composition obtained by the method of producing an aqueous ophthalmic composition according to the present embodiment, which contain the above-described components, the carbonic anhydrase inhibitor stably exists in each composition in the form of fine particles. Accordingly, these compositions have a low absorbance, and blurred vision caused by the administration thereof to the eye is effectively inhibited.

The dosage forms of the aqueous ophthalmic composition is not particularly restricted, and examples thereof generally include eye drop formulations.

### EXAMPLES

The embodiments of the present disclosure are described more specifically below by reference to examples. However, the scope of the present invention is not limited to these examples. Here, “%” and “part(s)” means “% by mass” and “part(s) by mass” unless otherwise specified.

### Examples 1 to 17 and Comparative Examples 1 to 7

Each aqueous ophthalmic composition was prepared in accordance with the below-described method.

### Preparation of Liquid A

First, an autoclave-sterilizable liquid A that contains a carbonic anhydrase inhibitor was prepared.

Table 1 shows the components and their amounts contained in the liquid A used in the production of the respective aqueous ophthalmic compositions of Examples and Comparative Examples.

Among the components of the liquid A shown in Table 1, raw materials other than brinzolamide as the carbonic anhydrase inhibitor were stirred in a beaker to obtain a solution in which aqueous components were dissolved.

Brinzolamide, the thus prepared solution, a stirring disk, and 306 g of yttria-stabilized zirconia beads (0.5-mm YTZ balls, manufactured by Nikkato Co., Ltd.) were placed in a bead mill container and stirred, whereby the liquid A was prepared.

### Sterilization of Liquid A

A lid was attached to the bead mill container that contains the liquid A, and sterilization was performed using an autoclave (SP200, manufactured by Yamato Scientific Co., Ltd.) at 123° C. for 40 minutes.

TABLE 1
(unit g)

| | | | | | | | | | HPMC | | |
|---|---|---|---|---|---|---|---|---|---|---|---|
| | | | TRITON | POLOXAMER | HPMC | HPMC | MC | MC | (METLOSE | MC | |
| | Brinzolamide | Tyloxapol | X-100 | 407 | (TC-5E) | (TC-5R) | (SM-4) | (SM-25) | 65SH-50) | (SM-100) | Water |
| Comparative | 4.284 | 0.102 | — | — | — | — | — | — | — | — | 29.614 |
| Example 1 | | | | | | | | | | | |
| Comparative | 4.284 | 0.102 | — | — | — | — | — | — | — | — | 29.614 |
| Example 2 | | | | | | | | | | | |
| Comparative | 4.284 | 1.428 | — | — | — | — | — | — | — | — | 28.288 |
| Example 3 | | | | | | | | | | | |
| Comparative | 4.284 | — | 0.102 | — | — | — | — | — | — | — | 29.614 |
| Example 4 | | | | | | | | | | | |
| Comparative | 4.284 | — | — | 0.102 | — | — | — | — | — | — | 29.614 |
| Example 5 | | | | | | | | | | | |
| Example 1 | 4.284 | — | — | — | 1.428 | — | — | — | — | — | 28.288 |
| Example 2 | 4.284 | — | — | — | — | 1.428 | — | — | — | — | 28.288 |
| Example 3 | 4.284 | — | — | — | — | — | 1.428 | — | — | — | 28.288 |
| Example 4 | 4.284 | — | — | — | — | — | — | 1.428 | — | — | 28.288 |
| Example 5 | 4.284 | — | — | — | — | — | — | — | 1.428 | — | 28.288 |
| Comparative | 4.284 | — | — | — | — | — | — | — | — | 1.428 | 28.288 |
| Example 6 | | | | | | | | | | | |
| Comparative | 4.284 | 0.102 | — | — | — | — | — | — | — | — | 29.614 |
| Example 7 | | | | | | | | | | | |
| Example 6 | 4.284 | — | — | — | 1.428 | — | — | — | — | — | 28.288 |
| Example 7 | 4.284 | — | — | — | 1.428 | — | — | — | — | — | 28.288 |
| Example 8 | 4.284 | — | — | — | 1.428 | — | — | — | — | — | 28.288 |
| Example 9 | 4.284 | — | — | — | 1.428 | — | — | — | — | — | 28.288 |
| Example 10 | 4.284 | — | — | — | 1.428 | — | — | — | — | — | 28.288 |
| Example 11 | 4.284 | — | — | — | 1.428 | — | — | — | — | — | 28.288 |
| Example 12 | 4.284 | — | — | — | 1.428 | — | — | — | — | — | 28.288 |
| Example 13 | 4.284 | — | — | — | 1.428 | — | — | — | — | — | 28.288 |
| Example 14 | 4.284 | 0.102 | — | — | 1.428 | — | — | — | — | — | 28.186 |
| Example 15 | 4.284 | — | 0.102 | — | 1.428 | — | — | — | — | — | 28.186 |
| Example 16 | 4.284 | — | — | 0.102 | 1.428 | — | — | — | — | — | 28.186 |
| Example 17 | 4.284 | — | — | — | 1.428 | — | — | — | — | — | 28.288 |

Preparation of Liquid B

Next, a liquid B to be used in a mixture was prepared.

Table 2 shows the components and their amounts contained in the liquid B used in the production of the respective aqueous ophthalmic compositions of Examples and Comparative Examples. As apparent from Table 2, the liquid B was a liquid consisting of only water in some cases.

The components of the liquid B shown in Table 2 were stirred and dissolved in a beaker, and the resultant was then subjected to filtration using a sterilization filter having a pore size of 0.2 μm. In the cases in which water was the sole component of the liquid B, water was directly used.

TABLE 2
(unit: g)

| | Polyoxyl | | | | |
|---|---|---|---|---|---|
| | 40 | CREMOPHOR | Potassium | Polyethylene | |
| | stearate | ELP | sorbate | glycol 6000 | Water |
| Comparative Example 1 | — | — | — | — | 17 |
| Comparative Example 2 | — | — | — | — | 17 |
| Comparative Example 3 | — | — | — | — | 17 |
| Comparative Example 4 | — | — | — | — | 17 |
| Comparative Example 5 | — | — | — | — | 17 |
| Example 1 | — | — | — | — | 17 |
| Example 2 | — | — | — | — | 17 |
| Example 3 | — | — | — | — | 17 |
| Example 4 | — | — | — | — | 17 |
| Example 5 | — | — | — | — | 17 |
| Comparative Example 6 | — | — | — | — | 17 |
| Comparative Example 7 | — | — | — | — | 17 |
| Example 6 | — | — | — | — | 17 |
| Example 7 | — | — | — | — | 14.96 |
| Example 8 | 0.0408 | — | — | 2.04 | 14.9192 |
| Example 9 | 0.102 | — | — | 2.04 | 14.858 |
| Example 10 | 0.204 | — | — | 2.04 | 14.756 |
| Example 11 | 0.408 | — | — | 2.04 | 14.552 |
| Example 12 | 0.816 | — | — | 2.04 | 14.144 |
| Example 13 | 0.102 | — | 0.204 | 2.04 | 14.654 |
| Example 14 | — | — | — | 2.04 | 14.96 |
| Example 15 | — | — | — | 2.04 | 14.96 |
| Example 16 | — | — | — | 2.04 | 14.96 |
| Example 17 | — | 0.102 | — | 2.04 | 14.858 |

Preparation of Mixture

After the sterilization, the bead mill container that contains the liquid A was taken out of the autoclave and the liquid A was stirred. Then, the liquid B obtained above or water was added to the bead mill container and stirred, whereby a mixture containing at least brinzolamide, a specific cellulose derivative, and water was obtained.

Wet Grinding

A stirring disk was connected to a bead mill apparatus (batch-type ready mill; vertical-type bead mill “RMB” manufactured by AIMEX Co., Ltd.) and bead-mill dispersion was performed at a rotation speed of 2,400 rpm for a treatment time of 4.5 hours while cooling the mixture-containing bead mill container with 10° C. cooling water, whereby a dispersion containing pulverized fine particles of brinzolamide as a solid component was obtained.

Preparation of Liquid C

Next, a liquid C used for dilution of the mixture containing fine particles of the carbonic anhydrase inhibitor obtained by the above wet grinding was prepared.

Table 3 shows the components and their amounts contained in the liquid C used in the production of the respective aqueous ophthalmic compositions of Examples and Comparative Examples.

The components of the liquid C shown in Table 3 were stirred and dissolved in a beaker and the pH of the resultant was adjusted, followed by 20-minute sterilization using an autoclave at 121° C., whereby a liquid C for dilution having a pH of 7.4 was obtained.

TABLE 3
(unit g)

| | | | | | | | pH-adjusting | | |
|---|---|---|---|---|---|---|---|---|---|
| | | | | | | | agent | | |
| | CARBOPOL | CARBOPOL | HPMC | Disodium | Sodium | | (hydrochloric acid, | | Total amount of |
| | 974PNF | 971PNF | (TC-5E) | edetate | chloride | Mannitol | sodium hydroxide) | Water | liquid C |
| Comparative Example 1 | 0.32 | — | — | 0.008 | 0.56 | 0.4 | q.s. | q.s. | 70 |
| Comparative Example 2 | — | 0.32 | — | 0.008 | 0.56 | 0.4 | q.s. | q.s. | 70 |
| Comparative Example 3 | — | 0.32 | — | 0.008 | 0.56 | 0.4 | q.s. | q.s. | 70 |
| Comparative Example 4 | — | 0.32 | — | 0.008 | 0.56 | 0.4 | q.s. | q.s. | 70 |
| Comparative Example 5 | — | 0.32 | — | 0.008 | 0.56 | 0.4 | q.s. | q.s. | 70 |
| Example 1 | — | 0.32 | — | 0.008 | 0.56 | 0.4 | q.s. | q.s. | 70 |
| Example 2 | — | 0.32 | — | 0.008 | 0.56 | 0.4 | q.s. | q.s. | 70 |
| Example 3 | — | 0.32 | — | 0.008 | 0.56 | 0.4 | q.s. | q.s. | 70 |
| Example 4 | — | 0.32 | — | 0.008 | 0.56 | 0.4 | q.s. | q.s. | 70 |
| Example 5 | — | 0.32 | — | 0.008 | 0.56 | 0.4 | q.s. | q.s. | 70 |
| Comparative Example 6 | — | 0.32 | — | 0.008 | 0.56 | 0.4 | q.s. | q.s. | 70 |
| Comparative Example 7 | — | — | 0.8 | 0.008 | 0.56 | 0.4 | q.s. | q.s. | 70 |
| Example 6 | 0.32 | — | — | 0.008 | 0.56 | 0.4 | q.s. | q.s. | 70 |
| Example 7 | — | 0.32 | — | 0.008 | 0.56 | 0.4 | q.s. | q.s. | 70 |
| Example 8 | — | 0.32 | — | 0.008 | 0.56 | 0.4 | q.s. | q.s. | 70 |
| Example 9 | — | 0.32 | — | 0.008 | 0.56 | 0.4 | q.s. | q.s. | 70 |
| Example 10 | — | 0.32 | — | 0.008 | 0.56 | 0.4 | q.s. | q.s. | 70 |
| Example 11 | — | 0.32 | — | 0.008 | 0.56 | 0.4 | q.s. | q.s. | 70 |
| Example 12 | — | 0.32 | — | 0.008 | 0.56 | 0.4 | q.s. | q.s. | 70 |
| Example 13 | — | 0.32 | — | 0.008 | 0.56 | 0.4 | q.s. | q.s. | 70 |
| Example 14 | — | 0.32 | — | 0.008 | 0.56 | 0.4 | q.s. | q.s. | 70 |
| Example 15 | — | 0.32 | — | 0.008 | 0.56 | 0.4 | q.s. | q.s. | 70 |
| Example 16 | — | 0.32 | — | 0.008 | 0.56 | 0.4 | q.s. | q.s. | 70 |
| Example 17 | — | 0.32 | — | 0.008 | 0.56 | 0.4 | q.s. | q.s. | 70 |

Dilution Step

To 70 g of the thus prepared liquid C, 10 g of the mixture (dispersion) containing the liquids A and B prepared in accordance with the amounts of components shown in Tables 1 and 2, which mixture had been subjected to wet grinding, was added, and the resultant was stirred to obtain an aqueous ophthalmic composition containing the respective components in the amounts shown in Tables 4 to 6.

In Comparative Example 6, since the mixture had a high viscosity and could not be wet ground, a dispersion could not be prepared.

In the following Examples and Tables, “2% viscosity” means the viscosity of a 2%-by-mass aqueous solution of each cellulose derivative at 20° C.

TABLE 4

| | Com- | Com- | Com- | | | | | |
|---|---|---|---|---|---|---|---|---|
| | parative | parative | parative | Comparative | Comparative | | | |
| | Example 1 | Example 2 | Example 3 | Example 4 | Example 5 | Example 1 | Example 2 | Example 3 |
| Component Concentration in liquid A | | | | | | | | |
| (in terms of the concentration | | | | | | | | |
| in the final aqueous composition, %) | | | | | | | | |
| Brinzolamide | 1.05 | 1.05 | 1.05 | 1.05 | 1.05 | 1.05 | 1.05 | 1.05 |
| Tyloxapol | 0.025 | 0.025 | 0.35 | — | — | — | — | — |
| TRITON X-100 | — | — | — | 0.025 | — | — | — | — |
| POLOXAMER 407 | — | — | — | — | 0.025 | — | — | — |
| HPMC (TC-5E, 2% viscosity: 2.5 to 3.5 mPa · s) | — | — | — | — | — | 0.35 | — | — |
| HPMC (TC-5R, 2% viscosity: 5.2 to 7.0 mPa · s) | — | — | — | — | — | — | 0.35 | — |
| MC (SM-4, 2% viscosity: 3.2 to 4.8 mPa · s) | — | — | — | — | — | — | — | 0.35 |
| Water | q.s. | q.s. | q.s. | q.s. | q.s. | q.s. | q.s. | q.s. |
| Component Concentration in liquid B | | | | | | | | |
| (in terms of the concentration | | | | | | | | |
| in the final aqueous composition, %) | | | | | | | | |
| Water | q.s. | q.s. | q.s. | q.s. | q.s. | q.s. | q.s. | q.s. |
| Component Concentration in liquid C | | | | | | | | |
| (in terms of the concentration | | | | | | | | |
| in the final aqueous composition, %) | | | | | | | | |
| Carboxyvinyl polymer (CARBOPOL 974PNF) | 0.4 | — | — | — | — | — | — | — |
| Carboxyvinyl polymer (CARBOPOL 971PNF) | — | 0.4 | 0.4 | 0.4 | 0.4 | 0.4 | 0.4 | 0.4 |
| HPMC (TC-5E, 2% viscosity: 2.5 to 3.5 mPa · s) | — | — | — | — | — | — | — | — |
| Disodium edetate | 0.01 | 0.01 | 0.01 | 0.01 | 0.01 | 0.01 | 0.01 | 0.01 |
| Sodium chloride | 0.7 | 0.7 | 0.7 | 0.7 | 0.7 | 0.7 | 0.7 | 0.7 |
| Mannitol | 0.5 | 0.5 | 0.5 | 0.5 | 0.5 | 0.5 | 0.5 | 0.5 |
| pH-adjusting agent (hydrochloric acid, sodium | q.s. | q.s. | q.s. | q.s. | q.s. | q.s. | q.s. | q.s. |
| hydroxide) | | | | | | | | |
| Water | q.s. | q.s. | q.s. | q.s. | q.s. | q.s. | q.s. | q.s. |
| Post-autoclave pH of liquid C | 7.4 | 7.4 | 7.4 | 7.4 | 7.4 | 7.4 | 7.4 | 7.4 |
| Absorbance (600 nm) | 1.452 | 1.453 | 1.963 | 2.084 | 1.556 | 0.462 | 0.439 | 0.525 |
| Evaluation (micronization of brinzolamide) | E | E | E | E | E | C | C | C |

TABLE 5

| | | | Comparative | Comparative | | | | |
|---|---|---|---|---|---|---|---|---|
| | Example 4 | Example 5 | Example 6 | Example 7 | Example 6 | Example 7 | Example 8 | Example 9 |
| Component Concentration in liquid A | | | | | | | | |
| (in terms of the concentration in the | | | | | | | | |
| final aqueous composition, %) | | | | | | | | |
| Brinzolamide | 1.05 | 1.05 | 1.05 | 1.05 | 1.05 | 1.05 | 1.05 | 1.05 |
| Tyloxapol | — | — | — | 0.025 | — | — | — | — |
| HPMC (TC-5E, 2% viscosity: 2.5 to 3.5 mPa · s) | — | — | — | — | 0.35 | 0.35 | 0.35 | 0.35 |
| HPMC (TC-5R, 2% viscosity: 5.2 to 7.0 mPa · s) | — | — | — | — | — | — | — | — |
| MC (SM-4, 2% viscosity: 3.2 to 4.8 mPa · s) | — | — | — | — | — | — | — | — |
| MC (SM-25, 2% viscosity: 20.0 to 30.0 mPa · s) | 0.35 | — | — | — | — | — | — | — |
| HPMC (METLOSE 65SH-50, 2% viscosity: | — | 0.35 | — | — | — | — | — | — |
| 40.0 to 60.0 mPa · s) | | | | | | | | |
| MC (SM-100, .2% viscosity: 80 to 120 mPa · s) | — | — | 0.35 | — | — | — | — | — |
| Water | q.s. | q.s. | q.s. | q.s. | q.s. | q.s. | q.s. | q.s. |
| Component Concentration in liquid B | | | | | | | | |
| (in terms of the concentration in the | | | | | | | | |
| final aqueous composition, %) | | | | | | | | |
| Polyoxyl 40 stearate | — | — | — | — | — | — | 0.01 | 0.025 |
| Polyethylene glycol 6000 | — | — | — | — | — | 0.5 | 0.5 | 0.5 |
| Water | q.s. | q.s. | q.s. | q.s. | q.s. | q.s. | q.s. | q.s. |
| Component Concentration in liquid C | | | | | | | | |
| (in terms of the concentration in the | | | | | | | | |
| final aqueous composition, %) | | | | | | | | |
| Carboxyvinyl polymer (CARBOPOL 974PNF) | — | — | — | — | 0.4 | — | — | — |
| Carboxyvinyl polymer (CARBOPOL 971PNF) | 0.4 | 0.4 | 0.4 | — | — | 0.4 | 0.4 | 0.4 |
| HPMC (TC-5E, 2% viscosity: 2.5 to 3.5 mPa · s) | — | — | — | 1 | — | — | — | — |
| Disodium edetate | 0.01 | 0.01 | 0.01 | 0.01 | 0.01 | 0.01 | 0.01 | 0.01 |
| Sodium chloride | 0.7 | 0.7 | 0.7 | 0.7 | 0.7 | 0.7 | 0.7 | 0.7 |
| Mannitol | 0.5 | 0.5 | 0.5 | 0.5 | 0.5 | 0.5 | 0.5 | 0.5 |
| pH-adjusting agent (hydrochloric acid, | q.s. | q.s. | q.s. | q.s. | q.s. | q.s. | q.s. | q.s. |
| sodium hydroxide) | | | | | | | | |
| Water | q.s. | q.s. | q.s. | q.s. | q.s. | q.s. | q.s. | q.s. |
| Post-autoclave pH of liquid C | 7.4 | 7.4 | 7.4 | 7.4 | 7.4 | 7.4 | 7.4 | 7.4 |
| Absorbance (600 nm) | 0.443 | 0.557 | dispersion | 1.429 | 0.462 | 0.456 | 0.334 | 0.375 |
| | | | could not be | | | | | |
| | | | prepared | | | | | |
| Evaluation (micronization of brinzolamide) | C | C | E | E | C | C | B | B |

TABLE 6

| | Example | Example | Example | Example | Example | Example | Example | Example |
|---|---|---|---|---|---|---|---|---|
| | 10 | 11 | 12 | 13 | 14 | 15 | 16 | 17 |
| Component Concentration in liquid A (in terms of the | | | | | | | | |
| concentration in the final aqueous composition, %) | | | | | | | | |
| Brinzolamide | 1.05 | 1.05 | 1.05 | 1.05 | 1.05 | 1.05 | 1.05 | 1.05 |
| Tyloxapol | | — | — | — | 0.025 | — | — | — |
| TRITON X-100 | — | — | — | — | — | 0.025 | — | — |
| POLOXAMER 407 | — | — | — | — | — | — | 0.025 | — |
| HPMC (TC-5E, 2% viscosity: 2.5 to 3.5 mPa · s) | 0.35 | 0.35 | 0.35 | 0.35 | 0.35 | 0.35 | 0.35 | 0.35 |
| Water | q.s. | q.s. | q.s. | q.s. | q.s. | q.s. | q.s. | q.s. |
| Component Concentration in liquid B (in terms of the | | | | | | | | |
| concentration in the final aqueous composition, %) | | | | | | | | |
| Polyoxyl 40 stearate | 0.05 | 0.1 | 0.2 | 0.025 | — | — | — | — |
| CREMOPHOR ELP | — | — | — | — | — | — | — | 0.025 |
| Potassium sorbate | — | — | — | 0.05 | — | — | — | — |
| Polyethylene glycol 6000 | 0.5 | 0.5 | 0.5 | 0.5 | 0.5 | 0.5 | 0.5 | 0.5 |
| Water | q.s. | q.s. | q.s. | q.s. | q.s. | q.s. | q.s. | q.s. |
| Component Concentration in liquid C (in terms of the | | | | | | | | |
| concentration in the final aqueous composition, %) | | | | | | | | |
| Carboxyvinyl polymer (CARBOPOL 971PNF) | 0.4 | 0.4 | 0.4 | 0.4 | 0.4 | 0.4 | 0.4 | 0.4 |
| Disodium edetate | 0.01 | 0.01 | 0.01 | 0.01 | 0.01 | 0.01 | 0.01 | 0.01 |
| Sodium chloride | 0.7 | 0.7 | 0.7 | 0.7 | 0.7 | 0.7 | 0.7 | 0.7 |
| Mannitol | 0.5 | 0.5 | 0.5 | 0.5 | 0.5 | 0.5 | 0.5 | 0.5 |
| pH-adjusting agent (hydrochloric acid, sodium | q.s. | q.s. | q.s. | q.s. | q.s. | q.s. | q.s. | q.s. |
| hydroxide) | | | | | | | | |
| Water | q.s. | q.s. | q.s. | q.s. | q.s. | q.s. | q.s. | q.s. |
| Post-autoclave pH of liquid C | 7.4 | 7.4 | 7.4 | 7.4 | 7.4 | 7.4 | 7.4 | 7.4 |
| Absorbance (600 nm) | 0.35 | 0.387 | 0.785 | 0.142 | 0.959 | 1.057 | 1.062 | 0.874 |
| Evaluation (micronization of brinzolamide) | B | B | D | A | D | D | D | D |

The details of the materials shown in Tables 1 to 6 are provided below.

Brinzolamide (carbonic anhydrase inhibitor; manufactured by Indoco Remedies Ltd.)

Tyloxapol (surfactant; manufactured by Sigma-Aldrich Co. LLC.)

TRITON (registered trademark) X-100 (trade name, manufactured by MP Biomedicals, LLC; surfactant)

POLOXAMER 407 (trade name: LUTROL F127, manufactured by BASF Corp.; polyoxyethylene-polyoxypropylene block copolymer)

HPMC TC-5E (hydroxypropylmethyl cellulose, trade name: TC-5E, manufactured by Shin-Etsu Chemical Co., Ltd.; specific cellulose derivative, 2% viscosity: 2.5 mPa·s to 3.5 mPa·s)

HPMC TC-5R (hydroxypropylmethyl cellulose, trade name: TC-5R, manufactured by Shin-Etsu Chemical Co., Ltd.; specific cellulose derivative, 2% viscosity: 5.2 mPa·s to 7.0 mPa·s)

MC SM-4 (methyl cellulose, trade name: SM-4, manufactured by Shin-Etsu Chemical Co., Ltd.; specific cellulose derivative, 2% viscosity: 3.2 mPa·s to 4.8 mPa·s)

MC SM-25 (methyl cellulose, trade name: SM-25, manufactured by Shin-Etsu Chemical Co., Ltd.; specific cellulose derivative, 2% viscosity: 20.0 mPa·s to 30.0 mPa·s)

HPMC METLOSE 65SH-50 (hydroxypropylmethyl cellulose, trade name: METLOSE 65SH-50, manufactured by Shin-Etsu Chemical Co., Ltd.; specific cellulose derivative, 2% viscosity: 40.0 mPa·s to 60.0 mPa·s)

MC SM-100 (methyl cellulose, trade name: SM-100, manufactured by Shin-Etsu Chemical Co., Ltd.; comparative cellulose derivative, 2% viscosity: 80 mPa·s to 120 mPa·s)

Polyoxyl 40 stearate (polyoxyethylene stearate, manufactured by Wako Pure Chemical Industries, Ltd.; polyoxyethylene fatty acid ester)

CREMOPHOR ELP (manufactured by BASF Corp.; polyoxyethylene castor oil)

Potassium sorbate (manufactured by Tokyo Chemical Industry Co., Ltd.)

Polyethylene glycol 6000 (manufactured by Wako Pure Chemical Industries, Ltd.)

Carboxyvinyl polymer (trade name: CARBOPOL (registered trademark) 974PNF, manufactured by The Lubrizol Corporation)

Carboxyvinyl polymer (trade name: CARBOPOL (registered trademark) 971PNF, manufactured by The Lubrizol Corporation)

Disodium edetate (manufactured by Wako Pure Chemical Industries, Ltd.)

Mannitol (trade name: MANNIT P, manufactured by Mitsubishi Shoji Foodtech Co., Ltd.)

Water (water for injection, manufactured by Hikari Pharmaceutical Co., Ltd.)

Measurement of Absorbance

The thus obtained aqueous ophthalmic compositions were each sampled and 10-fold diluted with water and, after adding the resultant to a glass cell having an optical path length of 10 mm, the absorbance was measured at a wavelength of 600 nm using a spectrophotometer (V-630BIO, manufactured by JASCO Corporation). The results thereof are shown in Tables 4 to 6. When the absorbance was 1.1 or less, it was determined that brinzolamide particles were sufficiently micronized. The evaluation criteria were as follows.

A: The absorbance was 0.2 or less. (Excellent micronization of the particles)

B: The absorbance was higher than 0.2 but 0.4 or less. (Favorable micronization of the particle)

C: The absorbance was higher than 0.4 but 0.7 or less. (Superior micronization of the particles than D)

D: The absorbance was higher than 0.7 but 1.1 or less. (The particles were micronized to a level in which blurred vision was inhibited)

E: The absorbance was higher than 1.1 (Insufficient micronization of the particles)

As shown in Tables 4 to 6, in all of the aqueous ophthalmic compositions obtained by the method of Examples, the brinzolamide particles contained as a carbonic anhydrase inhibitor were sufficiently micronized and the absorbance was low; therefore, these aqueous ophthalmic compositions are expected to show an effect of inhibiting the occurrence of blurred vision when administered to the eye.

Meanwhile, it is understood that, in Comparative Examples 1 to 5 in which a surfactant was allowed to coexist at the time of wet grinding and in Comparative Example 6 in which a cellulose derivative other than the specific cellulose derivative was incorporated, it was difficult to achieve the excellent effects as obtained by an embodiment of the present disclosure.

From the result of Examples 8 to 13, it is understood that the particles were more favorably micronized in a case in which the mixture contains a polyoxyethylene fatty acid ester at the time of wet grinding. Moreover, from the results of Example 13, it is understood that the particles were even more favorably micronized in a case in which the mixture further contains potassium sorbate.

From the result of Comparative Example 7, it is understood that such an effect of micronizing the brinzolamide particles could not be obtained in a case in which the specific cellulose derivative was added after the wet grinding process.

### Examples 18 to 22

Each aqueous ophthalmic composition was prepared in accordance with the below-described method.

### Preparation of Liquid A

First, an autoclave-sterilizable liquid A that contains a carbonic anhydrase inhibitor was prepared.

Table 7 shows the components and their amounts contained in the liquid A used in the production of the respective aqueous ophthalmic compositions of Examples 18 to 22.

Among the components of the liquid A shown in Table 7, raw materials other than brinzolamide as the carbonic anhydrase inhibitor were stirred in a beaker to obtain a solution in which aqueous components were dissolved.

Brinzolamide, the thus prepared solution, a stirring disk, and 306 g of yttria-stabilized zirconia beads (0.5-mm YTZ balls, manufactured by Nikkato Co., Ltd.) were placed in a bead mill container and stirred, whereby the liquid A was prepared.

### Moist Heat Sterilization of Liquid A

A lid was attached to the bead mill container that contains the liquid A, and moist heat sterilization was performed using an autoclave (SP200, manufactured by Yamato Scientific Co., Ltd.) at 123° C. for 40 minutes.

TABLE 7
Liquid A
(unit: g)

| | Example | Example | Example | Example | Example |
|---|---|---|---|---|---|
| | 18 | 19 | 20 | 21 | 22 |
| Brinzolamide | 4.284 | 2.142 | 2.142 | 2.142 | 2.142 |
| HPMC (TC-5E, | 1.428 | 0.714 | 0.714 | 0.642 | 0.581 |
| 2% viscosity: | | | | | |
| 2.5 to 3.5 | | | | | |
| mPa · s) | | | | | |
| Water | 28.288 | 14.144 | 14.144 | 14.215 | 14.277 |

Preparation of Liquid B

Next, a liquid B to be used in a mixture was prepared.

Table 8 shows the components and their amounts contained in the liquid B used in the production of the respective aqueous ophthalmic compositions of Examples 18 to 22.

The components of the liquid B shown in Table 8 were stirred and dissolved in a beaker, and the resultant was then subjected to filter sterilization using a sterilization filter having a pore size of 0.2 μm.

TABLE 8
Liquid B
(unit: g)

| | Examples 18 to 22 |
|---|---|
| Polyoxyl 40 stearate | 0.42 |
| CREMOPHOR ELP | 0.42 |
| Potassium sorbate | 0.84 |
| Polyethylene glycol 6000 | 8.4 |
| Water | 59.92 |

Preparation of Liquid C

Next, a liquid C was prepared.

Table 9 shows the components and their amounts contained in the liquid C used in the production of the respective aqueous ophthalmic compositions of Examples 18 to 22. The liquid C of the same composition was used in each of Examples 18, 19, 21, and 22. However, as described below, the order of adding the liquid C was different between Example 18 and Examples 19, 21, and 22.

The components of the liquid C shown in Table 9 were stirred and dissolved in a beaker and the pH of the resultant was adjusted, followed by 20-minute moist heat sterilization using an autoclave at 121° C., whereby a liquid C for dilution having a pH of 7.4 was obtained.

TABLE 9
Liquid C
(unit: g)

| | Example 18, | |
|---|---|---|
| | 19, 21, and 22 | Example 20 |
| Carboxyvinyl polymer (CARBOPOL | 0.64 | 0.16 |
| 971PNF) | | |
| Disodium edetate | 0.016 | 0.004 |
| Sodium chloride | 1.12 | 0.28 |
| Mannitol | 0.8 | 0.2 |
| pH-adjusting agent (hydrochloric acid, | q.s. | q.s. |
| sodium hydroxide) | | |
| Water | q.s. | q.s. |
| Total amount of Liquid C | 140 | 30 |

*The pH after the moist heat sterilization was 7.4 in all of these Examples.

Preparation of Mixture

After the sterilization, the bead mill container that contains the liquid A was taken out of the autoclave and the liquid A was stirred. Then, a liquid selected from the liquids B and C obtained above and water was added to the bead mill container in the amounts shown below.

Example 18: The liquid B was added in an amount of 17 g.

Examples 19, 21, and 22: The liquids B and C were added in amounts of 8.5 g and 25.5 g, respectively.

Example 20: The liquid B and water were added in amounts of 8.5 g and 25.5 g, respectively.

After adding the above-described liquid, the resultant was further stirred to obtain each mixture to be wet ground in Examples 18 to 22. The compositions of the mixtures to be wet ground in these Examples are shown in Table 10 below.

TABLE 10
Mixture
(unit: g)

| | Example | Example | Example | Example | Example |
|---|---|---|---|---|---|
| | 18 | 19 | 20 | 21 | 22 |
| Brinzolamide | 4.284 | 2.142 | 2.142 | 2.142 | 2.142 |
| HPMC (TC-5E, 2% viscosity: | 1.428 | 0.714 | 0.714 | 0.642 | 0.581 |
| 2.5 to 3.5 mPa · s) | | | | | |
| Polyoxyl 40 stearate | 0.102 | 0.051 | 0.051 | 0.051 | 0.051 |
| CREMOPHOR ELP | 0.102 | 0.051 | 0.051 | 0.051 | 0.051 |
| Potassium sorbet | 0.204 | 0.102 | 0.102 | 0.102 | 0.102 |
| Polyethylene glycol 6000 | 2.04 | 1.02 | 1.02 | 1.02 | 1.02 |
| Carboxyvinyl polymer | — | 0.117 | — | 0.117 | 0.117 |
| (CARBOPOL 971PNF) | | | | | |
| Disodium edetate | — | 0.0029 | — | 0.0029 | 0.0029 |
| Sodium chloride | — | 0.204 | — | 0.204 | 0.204 |
| Mannitol | — | 0.146 | — | 0.146 | 0.146 |
| pH-adjusting agent (hydrochloric | — | q.s. | — | q.s. | q.s. |
| acid, sodium hydroxide) | | | | | |
| Water | 42.84 | q.s. | 46.92 | q.s. | q.s. |
| Total amount of mixture | 51 | 51 | 51 | 51 | 51 |
| Time required for wetgrinding | 26.5 hr | 2.0 hr | 26.5 hr | 2.0 hr | 2.0 hr |

Wet Grinding

A stirring disk was connected to a bead mill apparatus (batch-type ready mill; vertical-type bead mill “RMB” manufactured by AIMEX Co., Ltd.) and bead-mill dispersion was performed at a rotation speed of 800 rpm while cooling the mixture-containing bead mill container with 10° C. cooling water, and 1 g of the thus obtained dispersion was sampled every 30 minutes.

To 1 g each of the sampled dispersions, the liquid C prepared in accordance with Table 9 was added in an amount of 7 g in Example 18 and in an amount of 3 g in each of Examples 19 to 22, and the resultant was stirred to obtain an aqueous ophthalmic composition containing the respective components in the amounts shown in Table 11.

TABLE 11
Aqueous ophthalmic composition

| | Examples | Example | Example |
|---|---|---|---|
| | 18 to 20 | 21 | 22 |
| Component Concentration in liquid A | | | |
| (in terms of the concentration in the | | | |
| final aqueous composition, %) | | | |
| Brinzolamide | 1.05 | 1.05 | 1.05 |
| HPMC (TC-5E, 2% viscosity: | 0.35 | 0.315 | 0.285 |
| 2.5 to 3.5 mPa · s) | | | |
| Water | q.s. | q.s. | q.s. |
| Component Concentration in liquid B | | | |
| (in terms of the concentration in the | | | |
| final aqueous composition, %) | | | |
| Polyoxyl 40 stearate | 0.025 | 0.025 | 0.025 |
| CREMOPHOR ELP | 0.025 | 0.025 | 0.025 |
| Potassium sorbate | 0.05 | 0.05 | 0.05 |
| Polyethylene glycol 6000 | 0.5 | 0.5 | 0.5 |
| Water | q.s. | q.s. | q.s. |
| Component Concentration in liquid C | | | |
| (in terms of the concentration in the | | | |
| final aqueous composition, %) | | | |
| Carboxyvinyl polymer | 0.4 | 0.4 | 0.4 |
| (CARBOPOL 971PNF) | | | |
| Disodium edetate | 0.01 | 0.01 | 0.01 |
| Sodium chloride | 0.7 | 0.7 | 0.7 |
| Mannitol | 0.5 | 0.5 | 0.5 |
| pH-adjusting agent (hydrochloric | q.s. | q.s. | q.s. |
| acid, sodium hydroxide) | | | |
| Water | q.s. | q.s. | q.s. |

Measurement of Absorbance and Time Required for Wet Grinding

The thus obtained aqueous ophthalmic compositions were each sampled and 10-fold diluted with water and, after adding the resultant to a glass cell having an optical path length of 10 mm, the absorbance was measured at a wavelength of 600 nm using a spectrophotometer (V-630BIO, manufactured by JASCO Corporation). The time required for the absorbance to be reduced to 0.2 or less was measured as the time required for wet grinding and shown in Table 10.

As shown in Table 10, as compared to Example 18, in Example 19 in which the mixture to be wet ground was 2-fold diluted with the liquid C that contains the carboxyvinyl polymer, the time required for wet grinding was drastically shortened. As compared to Example 18, in Example 20 in which the mixture to be wet ground was 2-fold diluted with water, the time required for wet grinding was the same as in Example 18.

Based on these comparisons of Examples 18 to 20, it is believed that, by incorporating a carboxyvinyl polymer in the mixture to be wet ground, the carboxyvinyl polymer functioned as a dispersant and the dispersion rate of brinzolamide was thereby improved.

By comparing Examples 19, 21, and 22, it was found that the same dispersion rate can be obtained even when the amount of HPMC is changed.

### Examples 23 and 24

In accordance with the methods described below, liquids A and B used in the production of aqueous ophthalmic compositions were prepared and the coarse dispersion of the mixture of the liquids A and B was evaluated.

### Preparation of Liquid A

First, an autoclave-sterilizable liquid A that contains a carbonic anhydrase inhibitor was prepared.

Table 12 shows the components and their amounts contained in the liquid A used in the production of the respective aqueous ophthalmic compositions of Examples.

Among the components of the liquid A shown in Table 12, raw materials other than brinzolamide as the carbonic anhydrase inhibitor were stirred in a beaker to obtain a solution in which aqueous components were dissolved.

After placing brinzolamide in a separate glass vessel, the thus prepared solution was added thereto and the resultant was mixed, whereby a liquid A was obtained.

TABLE 12
Liquid A
(unit: g)

| | Example 23 | Example 24 |
|---|---|---|
| Brinzolamide | 4.284 | 4.284 |
| HPMC (TC-5E, 2% viscosity: | 1.428 | 1.428 |
| 2.5 to 3.5 mPa · s) | | |
| Polyethylene glycol 6000 | — | 2.04 |
| Water | 28.288 | 26.248 |

Preparation of Liquid B

Next, a liquid B to be used in a mixture was prepared.

Table 13 shows the components and their amounts contained in the liquid B used in the production of the respective aqueous ophthalmic compositions of Examples.

The components of the liquid B shown in Table 13 were stirred and dissolved in a beaker, and the resultant was then subjected to filter sterilization using a sterilization filter having a pore size of 0.2 μm.

TABLE 13
Liquid B
(unit: g)

| | Example 23 | Example 24 |
|---|---|---|
| Polyoxyl 40 stearate | 0.102 | 0.102 |
| CREMOPHOR ELP | 0.102 | 0.102 |
| Potassium sorbate | 0.204 | 0.204 |
| Polyethylene glycol 6000 | 2.04 | — |
| Water | 14.552 | 16.592 |
| Coarse dispersion of mixture of liquids | B | A |
| A and B | | |

Moist Heat Sterilization of Liquid A

A lid was attached to the bead mill container that contains the liquid A, and moist heat sterilization was performed using an autoclave (SP200, manufactured by Yamato Scientific Co., Ltd.) at 123° C. for 40 minutes.

Coarse Dispersion Step

Subsequently, coarse dispersion was performed for homogenizing a mixture of the liquids A and B which was to be subjected to the wet grinding step. After the sterilization, when the glass vessel that contains the liquid A was taken out of the autoclave, aggregates of brinzolamide particles were observed in the liquid.

When the liquid B obtained above was added to the glass vessel that contains the liquid A and stirred using a magnetic stirrer, residual aggregates were visually observed in Example 23; however, no aggregate was observed and the resulting liquid was uniform in Example 24.

When the liquid of Example 23 containing residual aggregates was further stirred using a homogenizer (ULTRA-TURRAX T18-digital, manufactured by IKA K.K.) at 15,000 rpm, no aggregate was observed and the liquid was uniform.

The ease of coarse dispersion (coarse dispersibility) was evaluated based on the following criteria. The results thereof are shown in Table 13.

A: Simple stirring by a magnetic stirrer yielded a uniform liquid in which no aggregate was observed.

B: Stirring by a magnetic stirrer alone resulted in the observation of aggregates; however, further stirring by a homogenizer yielded a uniform liquid in which no aggregate was observed.

Wet Grinding

After placing the thus coarsely dispersed liquid and 306 g of sterilized yttria-stabilized zirconia beads (0.5-mm YTZ balls, manufactured by Nikkato Co., Ltd.) in a bead mill container, a stirring disk was connected to a bead mill apparatus (batch-type ready mill; vertical-type bead mill “RMB” manufactured by AIMEX Co., Ltd.), and bead-mill dispersion was performed at a rotation speed of 800 rpm while cooling the mixture-containing bead mill container with 10° C. cooling water. The liquid C of the same composition as the one used in Examples 18, 19, 21, and 22 was prepared, and 10 g of the thus obtained dispersion was added to 70 g of the liquid C, whereby an aqueous ophthalmic composition was obtained.

From a comparison between Examples 23 and 24, it was found that, when performing moist heat sterilization of a liquid containing a carbonic anhydrase inhibitor, a specific cellulose derivative, and water, the coarse dispersibility in the coarse dispersion step was improved by incorporating a polyethylene glycol into the liquid. This is presumably because the water-soluble polyethylene glycol was incorporated into the aggregates during the formation thereof after the moist heat sterilization and the aggregates of brinzolamide particles were thereby more easily dispersed during the coarse dispersion.

In Example 23 in which a polyethylene glycol was contained in the liquid B and this liquid B was mixed with the liquid A after moist heat sterilization, it is understood that the coarse dispersibility-improving effect provided by the addition of the polyethylene glycol was lower as compared to a case in which the polyethylene glycol was added to the liquid A.

The disclosures of Japanese Patent Application No. 2014-143640, filed Jul. 11, 2014, and Japanese Patent Application No. 2015-048743, filed Mar. 11, 2015, are incorporated herein by reference in their entirety.

All publications, patent applications, and technical standards mentioned in this specification are herein incorporated by reference to the same extent as if each individual publication, patent application, or technical standard was specifically and individually indicated to be incorporated by reference.

What is claimed is:

1. A method of producing an aqueous ophthalmic composition, the method comprising wet grinding a mixture that comprises a carbonic anhydrase inhibitor, a cellulose derivative, and water, wherein a 2%-by-mass aqueous solution of the cellulose derivative has a viscosity of 60 mPa·s or less at 20° C.

2. The method of producing an aqueous ophthalmic composition according to claim 1, wherein the carbonic anhydrase inhibitor is brinzolamide.

3. The method of producing an aqueous ophthalmic composition according to claim 1, wherein the cellulose derivative is at least one of hydroxypropylmethyl cellulose or methyl cellulose.

4. The method of producing an aqueous ophthalmic composition according to claim 1, wherein the mixture subjected to the wet grinding further comprises a carboxyvinyl polymer.

5. The method of producing an aqueous ophthalmic composition according to claim 1, wherein the mixture subjected to the wet grinding further comprises a polyoxyethylene fatty acid ester.

6. The method of producing an aqueous ophthalmic composition according to claim 5, wherein a content of the polyoxyethylene fatty acid ester is from 0.001% by mass to 0.1% by mass with respect to a total mass of the aqueous ophthalmic composition.

7. The method of producing an aqueous ophthalmic composition according to claim 5, wherein the polyoxyethylene fatty acid ester is polyoxyethylene monostearate.

8. The method of producing an aqueous ophthalmic composition according to claim 1, wherein the mixture subjected to the wet grinding further comprises at least one compound selected from the group consisting of sorbic acid and salts thereof.

9. The method of producing an aqueous ophthalmic composition according to claim 1, wherein the wet grinding is performed using a bead mill.

10. The method of producing an aqueous ophthalmic composition according to claim 1, wherein the method comprises adding a diluent containing water to the wet-ground mixture.

11. The method of producing an aqueous ophthalmic composition according to claim 1, wherein the method comprises subjecting at least some components of the mixture subjected to the wet grinding to moist heat sterilization prior to the wet grinding.

12. The method of producing an aqueous ophthalmic composition according to claim 11, wherein the components subjected to the moist heat sterilization comprise the carbonic anhydrase inhibitor, the cellulose derivative, the water, and polyethylene glycol.

13. An aqueous ophthalmic composition comprising a carbonic anhydrase inhibitor, a cellulose derivative, and water, wherein an absorbance of the aqueous ophthalmic composition at a wavelength of 600 nm and an optical path length of 1 mm is 1.1 or less and a 2%-by-mass aqueous solution of the cellulose derivative has a viscosity of 60 mPa·s or less at 20° C.

14. The aqueous ophthalmic composition according to claim 13, wherein the carbonic anhydrase inhibitor is brinzolamide.

15. The aqueous ophthalmic composition according to claim 13, wherein the cellulose derivative is at least one of hydroxypropylmethyl cellulose or methyl cellulose.

16. The aqueous ophthalmic composition according to claim 13, further comprising a carboxyvinyl polymer.

17. The aqueous ophthalmic composition according to claim 13, further comprising a polyoxyethylene fatty acid ester.

18. The aqueous ophthalmic composition according to claim 17, wherein a content of the polyoxyethylene fatty acid ester is from 0.001% by mass to 0.1% by mass with respect to a total mass of the aqueous ophthalmic composition.

19. The aqueous ophthalmic composition according to claim 17, wherein the polyoxyethylene fatty acid ester is polyoxyethylene monostearate.

20. The aqueous ophthalmic composition according to claim 13, further comprising at least one selected from the group consisting of sorbic acid and salts thereof.

21. The aqueous ophthalmic composition according to claim 13, further comprising polyethylene glycol.
